(19) United States
(12) Patent Application Publication
(10) Pub. No.: US 2021/0369903 A1
(43) Pub. Date: Dec. 2, 2021

(54) MEMS-Based Virus Treatment

(71) Applicant: Slobodan Petrovic, Rescue, CA (US)

(72) Inventor: Slobodan Petrovic, Rescue, CA (US)

(21) Appl. No.: 17/337,403

(22) Filed: Jun. 2, 2021

Related U.S. Application Data
(60) Provisional application No. 63/033,433, filed on Jun. 2, 2020.

(51) Int. Cl. A61L 2/26; A61M 31/00; A61M 1/00; A61L 2/04; A61L 2/10; A61L 2/03

(52) U.S. Cl. CPC A61L 2/26; A61M 31/00; A61M 1/77; A61L 2/04; A61L 2202/11; A61L 2/03; A61M 2202/0466; A61M 2210/0625; A61M 2205/0244; A61L 2/10

(57) ABSTRACT

A microelectromechanical device utilizing one or more micropumps embedded in a mouthguard for treatment and detection of viruses in a person's mouth. The micropump pumps saliva through the device where it can, for example, be treated with heat to destroy viruses in the saliva. In another embodiment the device can be used to detect the presence of virus in the saliva utilizing DNA PCR or chronoamperometry.

### CROSS-REFERENCES TO RELATED APPLICATION

This application claims the benefit of U.S. Provisional Patent Application No. 63/033,433 filed Jun. 2, 2020 which is incorporated by reference herein in its entirety.

### BACKGROUND

Viruses, including corona viruses, are present in the mucus of the mouth. COVID-19, as well as other viruses, can be detected inside a person's mouth and can be treated in the early stages of infection, before the incubation period is completed and the other body systems have been infected. They stay in the mucus for an amount of time before they are able to invade the rest of the body. There is a need for a device that can treat viruses before they have a chance to spread to the rest of the body or to others. The MEMS-based device reduces the virus load in the early stages of infection inside the mouth to defend against many respiratory diseases that start in the mouth. Besides COVID-19, other viruses as well as bacteria may be treated using the device.

### SUMMARY

Small fluid pumps known as micropumps are part of a class of microelectromechanical systems known by the acronym MEMS. MEMS devices are sensors and actuators on a scale with functional dimensions in the micrometer range. They are fabricated typically using silicon as the structural material, however many other types of materials can be used as well. These include, but are not limited to glass, ceramic, and polymer materials. MEMS sensors and actuators used in many applications with the best-known examples being accelerometers, pressure sensors, gyroscopes, digital micro-mirror arrays, thermal sensors, chemical and biological sensors. MEMS pumps, which is one of the components of this system, have been used in many different applications, such as drug delivery.

Various MEMS pumps are available, and they are classified as non-mechanical and mechanical. Mechanical or displacement MEMS pumps operate independent of fluid properties and offer higher flow rates and faster response times. The actuator mechanism types include the most common piezoelectric, but also electrostatic, thermopneumatic, shape-memory alloy, bimetallic, electromagnetic, and ionic conductive polymer films. Any of types of MEMS pump can be used in this invention. The non-mechanical MEMS pumps, using solution properties such as conductivity, electrokinetic, electroosmotic, electrowetting, electrophoretic, or electrohydrodynamic, can also be used in this invention. The preferred embodiment uses a piezoelectric micropump to cause saliva to move through the device.

The described MEMS-based device is embedded in a polymer or composite mouthguard and inserted over person's upper or lower teeth, or both. It has small capillaries at the edge of the mouthguard which become immersed in the oral mucus (i.e., saliva). In the most basic type of operation, when the pump is activated the diaphragm of the pump deflects up and down. When the diaphragm deflects up, the volume of the reservoir increases and mucus is drawn through the inlet capillaries through the inlet valve into the reservoir. When the diaphragm deflects down the volume of the reservoir decreases and mucus is pushed out through the outlet valve and the outlet capillary back to the oral cavity. Flowrates of saliva through the device are very low, in the range of 1-50 micro liters per minute, and therefore imperceptible to the person wearing the mouthguard device.

Embodiments of the device may configured to treat virus or to detect viruses. The destruction of viruses in the mucus during its passage through the MEMS device is preferably accomplished using heat. MEMS polysilicon heaters in the device create rapid temperature increase to destroy COVID-19 virus. The rise of temperature is rapid due to the small mass of silicon. Treatment with heat of around around 80° C. is effective at destroying viruses in the saliva. Other methods to treat virus include electromagnetic radiation, ultraviolet light, and application of voltage. The application of voltage to render viruses ineffective to cause infection is based on the fact that viruses (including COVID-19) have protein coating and proteins are sensitive to electrode potential. By application of appropriate voltage for a particular protein the reaction can be achieved to destroy the protein on the electrode surface, and thereby destroy the capacity of the virus to cause infection.

As the mucus from the surface of the mouth cavity is drawn into the device and cleansed from the viruses and the virus-free mucus is returned to the mouth, the cells in the mouth will excrete more mucus infected with pathogens such as COVID-19 or other viruses as a result of several process, the simplest one being diffusion based on concentration difference.

In another embodiment the device can be used to detect the presence of virus. One method to determine the presence of virus is through a DNA polymerase chain reaction. The micropump draws a sample into the reservoir where DNA polymerase chain reaction will take place. The micropump stops while the reaction occurs and a detector measures the result of the reaction providing a convenient method for a consumer to activate the analysis sequence on their own and see the results when the process is done.

An embodiment of the device can also be used to measure the presence of virus using chronoamperometry using electrodes. Since every protein has a different electrode decomposition potential, the resulting signal from the decomposition of virus at the electrode can be used to identify the type of protein and its concentration.

In another embodiment, the MEMS system can include a drug delivery system in the device to deliver certain drugs to the outlet of the virus-free mucus to replenish necessary components of the mucus or to deliver additional treatment that will be mixed with the rest of the mucus in the mouth.

The device can be used for a prolonged time to treat the virus or to prevent viruses from increasing its concentration in the mucus above the critical value and therefore preventing the disease.

In another embodiment, a testing/detection unit can be combined with the treatment unit. The mucus that is drawn into the device can be split into two flows. The first flow can undergo immediate treatment and virus removal, while the second flow can be used to perform detection or concentration determination for the virus. If the analysis shows no presence of the targeted virus, the treatment can be discontinued. The device with these multiple possibilities is ideal for prevention as well, for example during prolonged possible exposure to virus.

The device includes a small battery for power and electronics for controlling the device. The on-board power is used to drive the pump and the polysilicon heater, as well as the communication module. Several configurations of the complete system are possible. The main components include charge controller for the battery with USB, flow-meter control, thermistors, pump drive circuit, control circuit, and wireless communication control to enable Bluetooth connection with a smart phone or with the Internet of Things (IoT).

### DESCRIPTION OF THE DRAWINGS

FIG. 1 is a schematic of a mouthpiece with the MEMS-based device.

FIG. 2 is a schematic of a MEM-based device in a mouth.

FIG. 3 is an illustration of a MEMS-based device.

FIG. 4 is a schematic another embodiment of a MEMS-based device.

FIG. 5 is a blog diagram of a MEMS-based device.

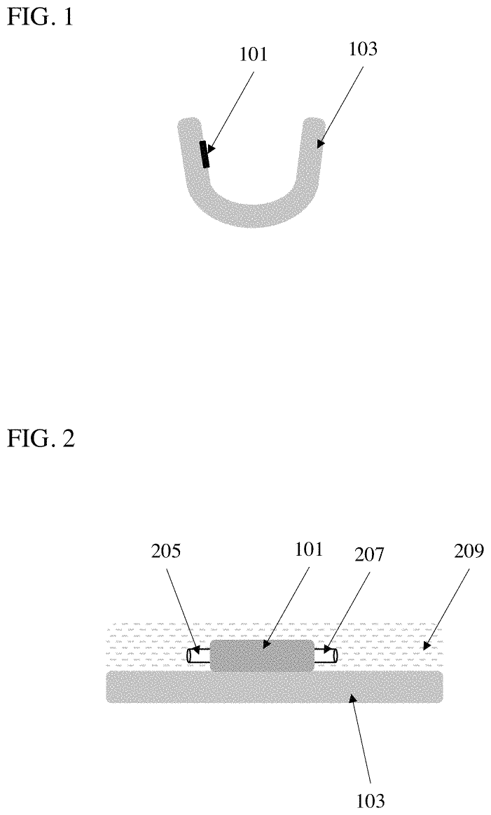

### DESCRIPTION

A MEMS-based device for detection and/or treatment 101 is attached to a mounting apparatus 103 such as a mouthpiece for insertion into one's mouth, typically on the teeth. The mounting apparatus 103 can be in any form that can fit over teeth to hold the elements of the device in place. It can be similar to an athletic mouth guard, or other device designed to fit in the mouth in a way that will hold the MEMS-based device 101 in place.

The mounting apparatus 103 can be made of any plastic material that can be molded into a an appropriate shape to fit over teeth while having the components embedded. The inlet 205 and outlet 207 to the MEMS-based device 101 come in contact with saliva 209 when the the mounting apparatus 103 is placed in a person's mouth. The material for the mounting apparatus 103 can be variety of plastics such as EVA (Ethylene vinyl acetate), PTFE (polytetrafluoroethylene), PVDF (polyvinylidene difluoride), or PVC (polyvinyl chloride). The mounting apparatus 103 can be formed to mount on all or some of a person's teeth, or in another fashion in the mouth, not necessarily over the teeth, to hold the MEMS-based treatment device 101 in place and in fluid contact with saliva.

With reference to FIG. 2, the MEMS-based device 101 has an inlet 205 and an outlet 207 that is in contact with saliva 209. The saliva 209 is drawn into the MEMS-based device 101 through the inlet 205, where treatment and/or detection can occur and then expelled back into the mouth through the outlet 207. The flow rates in this process are very low, in the range of 1-50 micro liters per minute, and therefore imperceptible to the person wearing the MEMS-based device 101.

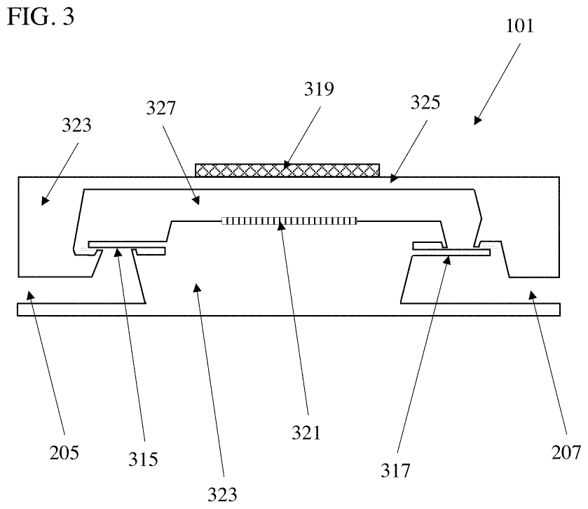

FIG. 3 shows an embodiment with a MEMS-based device 101 using single micropump having an overall length of less than one centimeter. The micropump has a body 323 with an inlet 205 and an outlet 207. The inlet valve 315 and outlet valve 317 permit the saliva to flow in only one direction. In the preferred embodiment the micropump is a piezoelectric micropump with a piezoelectric actuator 319. Voltage applied to the piezoelectric actuator 319 causes the diaphragm 325 to deflect up and down. The alternating deflection of the diaphragm 325 draws saliva in through the inlet valve 315 and pushes it out through the outlet valve 317. As saliva is drawn into the reservoir 327 the treatment or detection is effectuated by the functional component 321. For treatment of viruses with heat, the functional component 321 is a silicon heater set at a temperature of about 80 degrees C. For treatment viruses using ultraviolet light, the functional component 321 is a UV emitter.

For treatment viruses using voltage, the functional component 321 has two metal probes exposed to the saliva passing through the reservoir 319. Viruses, including corona viruses, have a protein skin that can be destroyed or denaturated with small electric currents. The small electric currents and low potential in a potentiostat creates an ionic current in the saliva. The ionic current drives the viruses towards the working electrode where they adhere. When a virus becomes adhered to the working electrode, the protein skin of the virus is destroyed or denaturated by the current rendering the virus incapable of causing infection. An electric potential of less than 1 volt is sufficient to create a current that will denaturate the protein skin of a virus, including the nucleocapsid protein and the spike protein found in the COVID-19 virus. Preferably, the voltage of the potentiostat of the functional component 321, can be adjusted. Adjustment of the voltage may be useful in targeting particular viruses of concern that are, or may be, in the saliva. The potentiostat may configured remotely using a wireless transceiver such as a Bluetooth transceiver 509 to connect with a smart phone or other computing device with an application designed to interface with the MEMS-based device 101.

The voltage necessary to adsorb protein on the surface of the functional component 321 is low, on the order of 0.2 to 0.4 V, but higher voltages up to 1 V may be used to overcome a potentially high ionic resistance in the saliva. The voltage can be tuned for a particular pathogen to values known to be effective for adsorption and charge transfer.

For treatment of viruses using electromagnetic radiation the functional component 321 is an emitter of energy in suitable wavelengths to react with the virus.

For detection of viruses through chonoamperomety, the functional component 321 will have a working electrode, a counter electrode, and a reference electrode connected to a potentiostate to measure the presence and concentration of virus.

The metal probes for virus detection can be made a number of metals such as gold, palladium, copper, or silver. The metal probes may be modified using special compounds containing receptors for particular proteins designed to enable more readily electron transfer to protein. The dimensions of the metal probes can typically be from 0.1 to 20 mm in length and 20 microns to 5 millimeters in width. It is also possible for the metal probes to be formed in an array, so called micro-electrodes, for added sensitivity. The metal probes can also be modified to embed small rotating electrodes using small electric motors. Rotation of the electrodes provides for better mass transport of mucus to the electrodes.

For detection of viruses through DNA polymerase chain reaction, the functional component 321 will have a heater that will be controlled to go through the sequence of thermal cycling necessary for the polymerase chain reaction and a detector to measure the results.

When the MEMS-based device 101 is connected wirelessly to a a smart phone or other computing device, data measured by the functional component 321 can be analyzed and/or transmitted through the Internet to medical services for analysis.

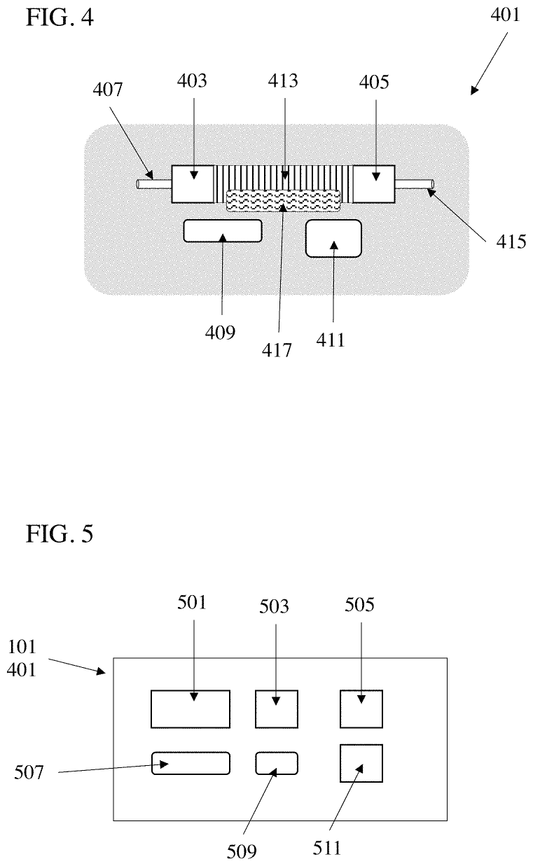

FIG. 4 illustrates an alternate embodiment of a MEMS-based device 401 having two micropumps, an inlet micropump 403 that draws saliva into a chamber 413 where treatment or detection can occur utilizing a functional component 417 and an outlet micropump 405 to return the saliva to the mouth. Saliva is draw through inlet 407 and then returned through the outlet 415. The micropumps, treatment and other functions are powered by a battery 409. A transceiver 411 allows for transmission of data out, and control, of the MEMS-based device 401. The chamber 413 has a functional component 417 suitable for the intended function of emitting electromagnetic radiation, ultraviolet light, heat, or voltage to destroy viruses. Or if the purpose of the MEMS-based device 401 is to detect viruses, the chamber 413 has a functional component 417 suitable for performing the DNA polymerase chain reaction and detection, or chronoamperometry.

FIG. 5 is a block diagram of the MEMS-based device 101 or 401. The MEMS-based device for virus detection and treatment in the mouth 101 may include a micropump assembly 501, a pump driver 503, an application-specific integrated circuit or ASIC 505, a battery 507, a wireless transceiver 509, and a drug delivery module 511.

A micropump assembly 501 contains the micropump(s) and the functional device. The micropumps are driven by a pump driver 503 that is controlled by an application-specific integrated circuit or ASIC 505. The functional component is also controlled by an ASIC 505. The MEMS-based device 101 or 401 communicates data and is controlled through a wireless transceiver 509. Everything is powered by a battery 507. The type of battery 507 can be, but not limited to, a primary (ZnO, Leclanche, or alkaline) battery, with the option to easily replace the battery. Alternatively, a rechargeable battery can be used, such as nickel metal hydride or lithium polymer, along with an appropriate mechanism for recharging the battery. The MEMS-based device 101 or 401 may also have a drug delivery device 511 to deliver medication into the saliva. The drug delivery device 511 would have a reservoir for the medication and its own micropump for dispensing the mediation during use.

What is claimed is:

1. An apparatus comprising:
a mounting apparatus adapted to fit over a person's teeth;
a micropump embedded in the mounting apparatus, the micropump having an inlet and an outlet;
said inlet and outlet are in contact with saliva when the mounting apparatus is placed in a person's mouth;
a functional component for treating virus.

2. The apparatus of claim 1 further comprising:
said micropump has a piezoelectric actuator.

3. The apparatus of claim 1 further comprising:
said functional component is a silicon heater.

4. An apparatus of claim 1 further comprising:
said functional component is a ultraviolet emitter.

5. The apparatus of claim 1 further comprising:
said functional component has a pair of electrodes for emitting voltage to the saliva.

6. The apparatus of claim 1 further comprising:
said functional component is an electromagnetic radiation emitter.
